(19) United States
(12) Plant Patent Application Publication
(10) Pub. No.: US 2014/0090124 P1
(43) Pub. Date: Mar. 27, 2014

(54) Tibouchina urvilleana plant named 'TB01'

(50) ;

(71) Applicant: Alan Haggo, Whenuapai (NZ)

(72) Inventor: Alan Haggo, Whenuapai (NZ)

(21) Appl. No.: 13/573,586

(22) Filed: Sep. 26, 2012

(51) Int. Cl. A01H 5/00

(52) U.S. Cl. PLT/263.1

(57) ABSTRACT

‘TB01’ is a distinctive variety of Tibouchina urvilleana which is characterized by unique foliage variegation ranging in color from yellow-green to pink.

The present application claims priority from a provisional U.S. application No. U.S. 61/626,450 filed Sep. 27, 2011, which is herein incorporated by reference.

### LATIN NAME OF THE GENUS AND SPECIES

The Latin name of the genus and species of the novel variety disclosed herein is Tibouchina urvilleana.

### VARIETY DENOMINATION

The inventive variety of Tibouchina disclosed herein has been given the variety denomination ‘TB01’.

### BACKGROUND OF THE INVENTION

The present invention relates to a new and distinct perennial variety of Tibouchina urvilleana, which has been given the variety denomination of ‘TB01’. Its market class is that of an ornamental plant. ‘TB01’ is intended for use in landscaping and as a decorative plant.

The Tibouchina urvilleana variety ‘TB01’ was selected in 2006 at a wholesale nursery near Whenuapai, New Zealand as a spontaneous genetic mutation among 500 plants of Tibouchina urvilleana ‘edwardsii’ (unpatented) production stock. ‘TB01’ was selected due to its unique foliage variegation with colors ranging from yellow-green to pink. ‘TB01’ was first propagated asexually by cuttings in Whenuapai, New Zealand and has since been asexually propagated by cuttings through four successive generations. The distinctive characteristics of the inventive ‘TB01’ variety are stable from generation to generation; clones of the variety produced by asexual reproduction maintain the distinguishing characteristics of the original plant.

### SUMMARY OF THE INVENTION

‘TB01’ is a distinctive variety of Tibouchina urvilleana which is characterized by unique foliage variegation ranging in color from yellow-green to pink, becoming more pronounced in cool weather.

### BRIEF DESCRIPTION OF THE FIGURES

FIG. 1 illustrates a typical ‘TB01’ plant during warm weather with flower.

FIG. 2 illustrates a ‘TB01’ plant with typical foliage color expression during cool weather.

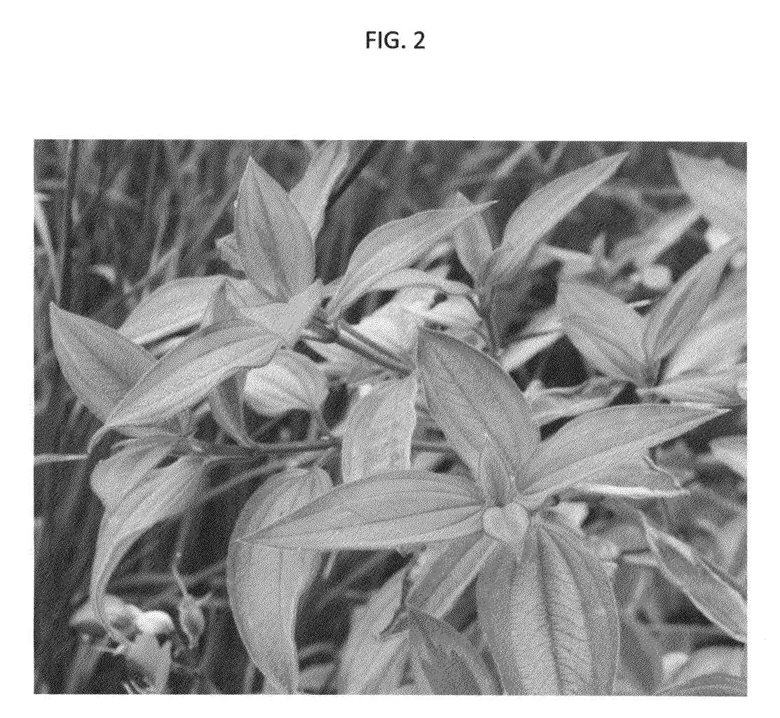

### BOTANICAL DESCRIPTION OF THE PLANT

The following is a detailed botanical description of a new and distinct variety of a Tibouchina urvilleana ornamental plant known as ‘TB01’. Plant observations were made on 14-month old plants grown in 200 mm nursery pots in New South Wales, Australia. Unless indicated otherwise, the descriptions disclosed herein are based upon observations made in October 2010 of mature ‘TB01’ plants grown in nursery pots.

Those skilled in the art will appreciate that certain characteristics will vary with older or, conversely, younger plants. ‘TB01’ has not been observed and other characteristics are given, it is to be understood that such characteristics are approximations or averages set forth as accurately as practicable. The phenotype of the variety may vary with variations in the environment such as season, temperature, light intensity, day length, cultural conditions and the like. Color notations are based on The Royal Horticultural Society Colour Chart, The Royal Horticultural Society, London, 2001 edition. Note that generic color descriptions such as ‘purple’ do not exist in the R.H.S. charts and the corresponding R.H.S. colors are quoted.

‘TB01’ is a perennial Tibouchina urvilleana plant which is a spontaneous mutation from a Tibouchina urvilleana ‘edwardsii’ stock plant. After its selection in 2006, ‘TB01’ was asexually propagated by cuttings. ‘TB01’ has a variegated leaf with colors ranging from yellow-green to pink and purple flowers displayed atop the foliage.

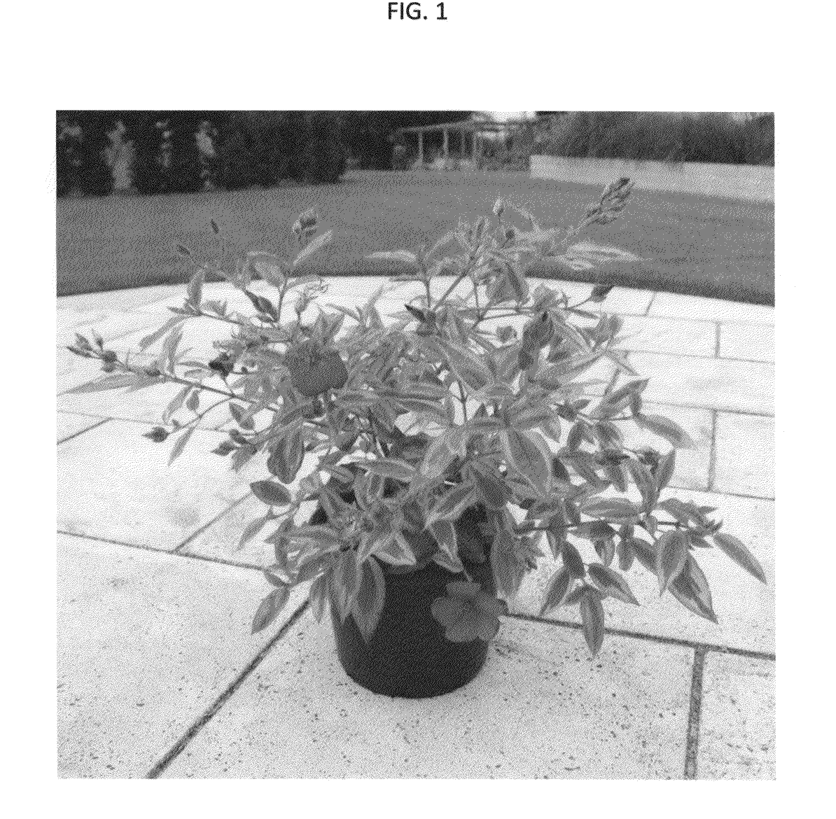

These features and other characteristics are apparent from the description provided below.
Growth habit, dimensions and color: ‘TB01’ is a tall plant forming an upright hedge habit. Average plant height is to 55 cm and average plant spread is 65 cm in a 14-month old plant grown in a 200 mm nursery pot in Sydney, New South Wales, Australia (observed October 2010). A representative ‘TB01’ plant is shown in FIG. 1.
Lateral branches: Quantity per plant: many; greater than 10. Length: Approximately 16 to 20 cm on average. Diameter: Approximately 5 to 7 mm. Internode length: Approximately 29 mm on average. Strength: Strong. Texture: Pubescent. Color: Immature: 146C. Mature: Woody, 199D.
Foliage: The leaf has an average width of 37 mm and reaches an average length of 90 mm, including petiole (which is approximately 10 to 15 mm in length and 3 to 4 mm wide). Leaves are opposite and simple. The leaf shape is lanceolate, leaf base is obtuse, leaf margin is entire, leaf apex is acute. The texture of both the upper and lower leaf surfaces texture is coarse and pubescent. The leaf venation pattern is parallel; the color does not contrast significantly from that of the surrounding foliage. Foliage Color: Young foliage, upper surface, during warm weather: multi-color variegation beginning with approximately Red-Purple (RHS 61B) at the margin moving to approximately Yellow-Green (RHS 151C) on the outer half of the leaf blade and then to Green (RHS 137A) near the midrib. Young foliage, lower surface, during warm weather follows the same variegation pattern as the upper leaf surface only colors are slightly muted: margin corresponds to 61C, moving to approximately 151D and then approximately 137C at the midrib. Mature foliage, upper surface, during warm weather: variegated; Yellow-Green (RHS 150B) on the outer half of the leaf blade to Green (RHS 135C) on the interior half of the leaf blade. Mature foliage, lower surface, during warm weather: also variegated and corresponding to approximately Yellow-Green (RHS 150C) transitioning to approximately Green (RHS 135C) on the interior of the leaf. Young foliage, upper surface, during cool weather: leaf margin and outer third of leaf blade corresponding to approximately Red (RHS 52A) with the interior of leaf being a blend of red and green pigments corresponding to approximately Red-Purple (RHS 59A). Young foliage, lower surface, during cool weather is similar to upper surface only slightly muted: near leaf margin and outer third of leaf the color corresponds most closely to RHS 52C with the interior of the leaf corresponding to approximately RHS 59C. Mature foliage, upper surface, during cool weather: variegated; at the margin and outer half there is a blend of both Orange-Red (approximately RHS 34C) and Yellow-Green (approximately RHS 150B) transitioning to approximately Yellow-Green (RHS 151B) on the interior of the leaf. Mature foliage, lower surface, during cool weather: similar to upper surface only slightly muted; RHS 34D at the margin and outer leaf transitioning to approximately RHS 151C. Venation: Midribs (at all stages) do not contrast significantly with the surrounding leaf blade color. 147C. Petiole, young and mature, during warm weather is Yellow-Green (approximately RHS 151C). Petiole, young and mature, during cool weather is Red (approximately RHS 53B)
Roots: The roots of ‘TB01’ are colored white and appear fibrous and freely branched, similar to other Tibouchina urvilleana.
Flowers:
   Flower type and habit.—‘TB01’ has a flower typical of the parent plant. Rounded flowers arranged in terminal panicles, typically three to five flowers per panicle, facing upward and outward. Individual flowers typically persist for a period of no more than two days.
   Natural flowering season.—Summer through Fall.
   Fragrance.—None.
   Flowers.—Diameter: Approximately 85 mm.
   Flower buds.—Length: Approximately 25 to 30 mm. Diameter: About 10 to 15 mm. Shape: Ovoid. Color: Red (RHS 46A).
   Petals.—Arrangement: Five petals in a single whorl. Length: Approximately 35 to 40 mm. Width: Apex, approximately 20 to 25 mm; base, about 5 mm. Shape: Obovate. Apex: Rounded. Base: Cuneate. Margin: Entire. Texture, upper and lower surfaces: Smooth. Aspect: Concave towards base, reflexed towards apex. Color: When opening: 83A. When opening, lower surface: 82A. Fully opened, upper surface: Violet (RHS 82A). Fully opened, lower surface: Violet (RHS 82B).
   Sepals.—Arrangement: Five sepals in a single whorl. Length: Approximately 10 to 15 mm. Width: Approximately 5 mm. Shape: Lanceolate. Apex: Acute. Base: Cuneate. Texture, upper and lower surfaces: Pubescent. Color, upper and lower surfaces: a blend of Red (RHS 46A) and Yellow-Green (RHS 151D).
   Peduncles.—Length: Approximately 30 to 35 mm. Diameter: Approximately 5 to 7 mm. Strength: Strong. Texture: Pubescent. Color is a blend of Red (RHS 46A) and Yellow-Green (RHS 151D).
   Reproductive organs.—Androecium: Quantity of Stamen per flower: usually ten. Filament length: Approximately 20 mm. Filament color: 82B. Anther length: About 15 mm. Anther color: Approximately Violet (RHS 83C). Pollen: None observed. Gynoecium: Pistils: Quantity per flower: One with five locules. Pistil length: approximately 35 mm. Style length: About 30 mm. Style color: 82A. Stigma shape: Round. Stigma color: 155C. Ovary color: 145B.
   Seed/fruit.—Seed production has not yet been observed.
Comparison of TB01 with other varieties of Tibouchina urvilleana: ‘TB01’ is the only known variegated form of T. urvilleana known to the breeder. Variegated forms exist among other species of Ticouchina. However, significant difference in plant habit and leaf shape make them unfit for comparators. ‘TB01’ is characterized by a variegated leaf blade with color ranging from yellow-green to pink as compared with the parent T. urvilleana ‘edwardsii’ which has no variegation of the leaf blade. This unique foliage variegation makes ‘TB01’ a desirable ornamental plant suited for mass production for pot and landscape use.
Asexual reproduction: After its initial discovery, subsequent cuttings were taken from the original T. urvilleana ‘TB01’ plant and transplanted into 90 mm×50 mm×50 mm propagation trays to increase stock. Once these trays were fully rooted, these young plants were transferred into 140 mm pots to allow for testing of genetic stability and also for further field trials and testing. After several subsequent generations of cuttings, ‘TB01’ was observed to retain foliage variegation that was noted in the original ‘TB01’ mutation. Cuttings of ‘TB01’ root within 2 weeks. Potting mix used contained 50% sand and 50% peat.
Environmental tolerances: ‘TB01’ has typical shade tolerance for the species. The winter hardiness of ‘TB01’ is at least to USDA Zone 9a in the Southeastern United States, and evaluation of winter hardiness is ongoing. Like the parent plant, ‘TB01’ has some drought tolerance but benefits regular watering. ‘TB01’ does well in sandy soils, but also tolerates heavy, clay-type soils well.
Disease resistance: ‘TB01’ has typical resistance to fungal and bacterial pathogens compared to other similar Tibouchina urvilleana cultivars.

That which is claimed is:

1. A new and distinct variety of Tibouchina urvilleana plant named ‘TB01’, substantially as described and illustrated herein.
